(19) United States
(12) Patent Application Publication
(10) Pub. No.: US 2013/0331607 A1
(43) Pub. Date: Dec. 12, 2013

(54) PROCESS FOR PREPARING FORMIC ACID

(71) Applicants: Thomas Schaub, Neustadt (DE); Sabine Huber, Bobenheim-Roxheim (DE); Rocco Paciello, Bad Durkheim (DE); Anton Meier, Birkenheide (DE); Giuseppe Fachinetti, Pisa (IT); Debora Preti, Nodica-Vecchiano (pi) (IT)

(72) Inventors: Thomas Schaub, Neustadt (DE); Sabine Huber, Bobenheim-Roxheim (DE); Rocco Paciello, Bad Durkheim (DE); Anton Meier, Birkenheide (DE); Giuseppe Fachinetti, Pisa (IT); Debora Preti, Nodica-Vecchiano (pi) (IT)

(73) Assignee: BASF SE, Ludwigshafen (DE)

(21) Appl. No.: 13/913,644

(22) Filed: Jun. 10, 2013

Related U.S. Application Data
(60) Provisional application No. 61/657,938, filed on Jun. 11, 2012.

(51) Int. Cl. C07C 51/15

(52) U.S. Cl. CPC C07C 51/15
USPC 562/609

(57) ABSTRACT

A process for preparing formic acid by hydrogenation of carbon dioxide in the presence of a tertiary amine (I), a diamine (II), a polar solvent and a catalyst comprising gold at a pressure of from 0.2 to 30 MPa abs and a temperature of from 0 to 200° C., wherein the catalyst is a heterogeneous catalyst comprising gold.

### RELATED APPLICATIONS

This patent application claims the benefit under 35 U.S.C. §119(e) of U.S. Provisional Application Ser. No. 61/657,938 filed on Jun. 11, 2012, incorporated in its entirety herein by reference.

### BACKGROUND OF THE INVENTION

The present invention relates to a process for preparing formic acid by hydrogenation of carbon dioxide in the presence of a tertiary amine (I), a diamine (II), a polar solvent and a heterogeneous catalyst comprising gold at a pressure of from 0.2 to 30 MPa abs and a temperature of from 20 to 200° C.

Formic acid is an important and versatile product. It is used, for example, for acidification in the production of animal feeds, as preservative, as disinfectant, as auxiliary in the textile and leather industry, as a mixture with its salts for deicing aircraft and runways and also as synthetic building block in the chemical industry.

The commonest process at present for the preparation of formic acid seems to be the hydrolysis of methyl formate. The aqueous formic acid obtained by hydrolysis is subsequently concentrated, for example by use of an extracting agent such as, for example, a dialkylformamide.

In addition, it is known that formic acid can also be obtained by thermal cleavage of compounds of formic acid and a tertiary nitrogen base. These compounds are in general acid ammonium formates of tertiary nitrogen bases, in which the formic acid has reacted beyond the stage of classic salt formation with the tertiary nitrogen bases to give stable addition compounds bridged via hydrogen bridge bonds. These compounds can be prepared in various ways, such as (i) by direct reaction of tertiary amine with formic acid, (ii) by hydrolysis of methyl formate to form formic acid in the presence of the tertiary amine or with subsequent extraction of the hydrolysis product with the tertiary amine or (iii) by catalytic hydration of carbon monoxide or hydrogenation of carbon dioxide to form formic acid in the presence of the tertiary amine. The latter process of catalytic hydrogenation of carbon dioxide has the particular attraction that carbon dioxide is available in large quantities and is flexible in terms of source.

The fundamental work on the catalytic hydrogenation of carbon dioxide to form formic acid was carried out as early as the 1970s and 1980s. The processes of BP Chemicals Ltd. filed as the patents EP 0 095 321 A, EP 0 151 510 A and EP 0 181 078 A may be considered to result therefrom. All three documents describe the hydrogenation of carbon dioxide in the presence of a homogeneous catalyst comprising a transition metal of transition group VIII (groups 8, 9, 10), a tertiary amine and a polar solvent to form an adduct of formic acid and the tertiary amine. As preferred homogeneous catalysts, EP 0 095 321 A and EP 0 181 078 A mention ruthenium-based and EP 0 151 510 A rhodium-based complex catalysts. Preferred tertiary amines are C₁-C₁₀-trialkylamines, in particular the short-chain C₁-C₄-trialkylamines, and also cyclic and/or bridged amines such as 1,8-diazabicyclo[5.4.0]undec-7-ene, 1,4-diazabicyclo[2.2.2]octane, pyridine or picolines. The hydrogenation is carried out at a carbon dioxide partial pressure of up to 6 MPa (60 bar), a hydrogen partial pressure of up to 25 MPa (250 bar) and a temperature from about room temperature to 200° C.

P. G. Jessop, Homogeneous Hydrogenation of Carbon Dioxide, in “The Handbook of Homogeneous Hydrogenation”, Ed.: J. G. de Vries and C. J. Elsevier, Volume 1, 2007, Wiley-VCH Verlag GmbH & Co KGaA, pages 489 to 511 presents an overview on the typically used catalysts for the hydrogenation of carbon dioxide. The focus is directed to homogeneous catalysts based on elements of group VIII (groups 8, 9, 10) of the periodic table, namely Fe, Ni, Ru, Rh, Pd and Ir, but Mo and Ti are also mentioned as suitable elements.

It is crucial for an economic process that the used hydrogenation catalyst has to be removed from the product stream and recycled back into the hydrogenation reactor, because losses of catalyst would require compensation by addition of new catalyst. Another reason for the removal of the catalyst from the product stream is, that hydrogenation catalysts also catalyze the decomposition of formic acid into carbon dioxide and hydrogen, which would lead to losses of formic acid in the process. The decomposition of formic acid in the presence of hydrogenation catalysts was, for example, investigated by C. Fellay et al. and published in Chem. Eur. J. 2009, 15, pages 3752 to 3760.

WO 2010/149,507 teaches a way to solve this problem by carrying out the homogeneously catalyzed hydrogenation in the presence of a tertiary amine and a polar solvent to form two liquid phases, in which one phase is enriched with the polar solvent and the formed formic acid/amine adduct, and the other phase is enriched with tertiary amine and the homogeneous catalyst, whereby the latter one containing the homogeneous catalyst is recirculated to the hydrogenation reactor. High boiling amines like Trihexylamine were described in this work as the amine in the hydrogenation. These long-chain Trialkylamines has the advantage that formic acid can directly distilled of from the amine. Nevertheless, the handling of the homogeneous catalysts is a disadvantage of their use.

Heterogeneous catalysts are known to be generally much more easier separated from the reaction products. Unfortunately, neither finely devided metal particles nor conventional metal-based supported catalysts with the metals known from the homogeneous carbon dioxide hydrogenation catalysts show suitable activities and selectivities in the hydrogenation of carbon dioxide.

However, A. Baiker discloses in Appl. Organometal. Chem. 14, 2000, pages 751 to 762 the hydrogenation of carbon dioxide to formic acid derivatives in the presence of immobilized homogeneous catalysts. These specific catalysts are synthesized by functionalizing group VIII (groups 8, 9, 10) transition metal complexes, such as [Ru(PR₃)₃Cl₂], with bifunctional silylether-modified phosphines, like Ph₂P(CH₂)₂Si(OEt)₃ or (CH₃)₂P(CH₂)₂Si(OEt)₃, and reacting them with Si(OEt)₄ (triethoxysilan), obtaining an immobilized transition metal-based silica hybrid gel complex catalyst.

Years later, Z. Zhang et al. published in ChemSusChem 2009, 2, pages 234 to 238 the hydrogenation of carbon dioxide to a formic acid/amine adduct in the presence of 1,3-di(N,N-dimethylaminoethyl)-2-methylimidazolium trifluoromethansulfonate, as amine and ionic liquid, and a specific immobilized homogeneous Ruthenium complex catalyst. The catalyst was prepared by treating silica with (EtO)₃Si(CH₂)₃Cl in toluene and thioacetamide in water, reacting the resulting product with RuCl₃.3H₂O in ethanol, and mixing the formed catalyst precursor with PPh₃ to obtain the immobilized Ru-based complex catalyst, expressed as “Si”—(CH₂)₃NH(CSCH₃)—{RuCl₃(PPh₃)}.

One disadvantage of these immobilized homogeneous catalysts mentioned-above is the complex and elaborate multi step synthesis. Also this catalysis was just described with NEt₃ (triethylamine) as the base. It is known from literature (e.g. EP 0 095 321 A, EP 0 151 510 A and EP 0 181 078 A), that formic acid cannot be separated thermally from this amine by distillation due to the formation of stable azeotropes. Therefore a additional, elaborated step has to be done in which the low boiling NEt₃ must be exchanged by a high boiling amine (e.g. alkylimidazoles), from which the formic acid can then be distilled of.

Currently, D. Preti et al. published in Angew. Chem. Int. ed. 50, 2011, pages 12551-12554 a system with a heterogeneous gold catalyst for the direct synthesis of formic acid in neat NEt₃ as the solvent and the base. They used the commercial available simple Aurolite Catalysts (Au on TiO₂). The reaction was carried out in an autoclave which was charged with pure NEt₃ and pressurized with carbon dioxide and hydrogen to 180 bar at 40° C. In order to obtain the free formic acid a base exchange of the NEt₃ by the high boiling NHex₃ (trihexylamine) is carried out. The Formic acid salt of NHex₃ obtained in the base exchange step is afterwards thermally cleaved and the free formic acid can afterwards be distilled off.

The above mentioned process also has the disadvantage that the low boiling triethylamine (NEt₃) cannot be separated from the formic acid, so that a base exchange step is required. This step requires additional energy in the production and is also leading to a higher investment for a production plant.

It was an object of the present invention to discover a process for preparing formic acid by hydrogenation of carbon dioxide, which does not have the above-mentioned disadvantages of the prior art or suffers from them only to a significantly reduced extent and allows concentrated formic acid to be obtained in a high yield and high purity.

Furthermore, the process should be able to be carried out in a simple manner or at least a simpler manner than described in the prior art, for example by means of a different, simpler process concept, simpler process stages, a reduced number of process stages or simpler apparatuses. Losses of valuable catalyst should be reduced and also the separation and recycling of the catalyst from the product phase should be simple. In addition, the process should also be able to be carried out with a low consumption of energy. In a preferred embodiment it is an object of the present invention to discover a process for preparing formic acid without the need of a base exchange step.

### A SUMMARY OF THE INVENTION

We have accordingly found a process for preparing formic acid by hydrogenation of carbon dioxide in the presence of a tertiary amine (I), a diamine (II), a polar solvent and a catalyst comprising gold at a pressure of from 0.2 to 30 MPa abs and a temperature of from 0 to 200° C., wherein the catalyst is a heterogeneous catalyst comprising gold.

### A BRIEF DESCRIPTION OF THE FIGURE

FIG. 1 shows a schematic block diagram of an embodiment of the process of the invention.

### A DETAILED DESCRIPTION OF THE INVENTION

The heterogeneous catalyst comprising gold to be used in the hydrogenation of carbon dioxide can be present in various types. In general, it can be gold itself or gold supported by a support material. In case of being gold itself, preferably gold black is used, but also other types like supported gold nanoparticles are possible. In addition, gold alloys, i.e. Au-M on supports can also be used, where M can be a precious metal like Pd or Pt as well as other kind of metals such as Ag or Cu. Also different metal promoters can be used in one and the same catalyst.

Preferably, the heterogeneous catalyst comprising gold is a supported catalyst. As support, various types of materials might be used, including but not limited to inorganic oxides, graphite, polymers or metals. In case of inorganic oxides, silicon dioxide, aluminium oxide, zirconium oxide, magnesium oxide and/or titanium oxide are preferred, but also other inorganic oxides are applicable. Particularly preferred are magnesium oxide, aluminium oxide, silica oxide, gallium oxide, zirconium oxide, ceria oxide and/or titanium oxide as support. Furthermore, mixtures of different inorganic oxides can also be used. The heterogeneous catalyst can be used in various geometric shapes and sizes, for example from powder to shaped material. In the case of a fixed-bed catalyst, use is made of, for example, pellets, cylinders, hollow cylinders, spheres, rods or extrudates. Their average particle diameter is generally from 1 to 10 mm. In case of metals or polymers as support, also meshes or knitted and crocheted wires or fabrics are applicable. Preferred is a process, wherein the supported heterogeneous catalyst comprises silicon dioxide, aluminium oxide, zirconium oxide, magnesium oxide and/or titanium oxide as support.

In case of a supported catalyst, the heterogeneous catalyst generally comprises 0.01 to 50 wt.-% (% by weight), preferably 0.1 to 20 wt.-% and particularly preferably 0.1 to 5 wt.-% gold, based on the total mass of the supported catalyst. In case of a non-supported catalyst, the amount of gold is generally from 0.01 to 100 wt.-%, based on the total weight of the catalyst.

Suitable heterogeneous catalysts comprising gold are commercially available or can be obtained by treatment of the support with a solution of a gold component or co-precipitation and subsequent drying, heat treatment and/or calcination by known methods.

Irrespective of whether the heterogeneous catalyst comprising gold is a supported or non-supported catalyst and irrespective of whether it additionally contains further metals (e.g. in the form of gold alloys), the heterogeneous catalyst comprising gold generally comprises gold containing particles with a diameter of 0.1 to 50 nm, measured by X-ray diffraction spectroscopy. Additionally, it may also contain particles with a diameter of less than 0.1 nm and/or more than 50 nm.

Furthermore and also irrespective of whether the heterogeneous catalyst comprising gold is a supported or non-supported catalyst and irrespective of whether it additionally contains further metals (e.g. in the form of gold alloys), the heterogeneous catalyst comprising gold generally exhibits a BET surface of ≧1 m²/g and ≦1000 m²/g, determined in accordance with DIN ISO 9277. It preferably exhibits a BET surface of ≧10 m²/g and ≦500 m²/g.

The volume of the heterogeneous catalyst comprising gold in the reactor (hydrogenation reactor) is generally between 0.1 and 95% of the reactor volume, whereby the catalyst's volume is calculated by the catalyst's mass divided by its bulk density.

The tertiary amine (I) to be used in the hydrogenation of carbon dioxide in the process of the invention preferably comprises at least 12 carbon atoms. It is preferably an amine of the general formula (I)

NR¹R²R³ (I)

where the radicals R¹ to R³ are identical or different and are each, independently of one another, an unbranched or branched, acyclic or cyclic, aliphatic, araliphatic or aromatic radical having from 1 to 46 carbon atoms, preferably from 1 to 18 carbon atoms, but in total R¹ to R³ together having at least 12 carbon atoms and not more than 48 carbon atoms, where individual carbon atoms can also be substituted, independently of one another, by a hetero group selected from the groups consisting of —O— and >N— or two or all three radicals can also be joined to one another to form a chain comprising at least four atoms in each case. Preference is given to at least one of the radicals bearing two hydrogen atoms on the alpha-carbon atom.

Examples of suitable tertiary amines (I) are:
Tributylamines (including tri-n-butylamine, tri-iso-butylamine and mixed isomers), tripentylamines (including tri-n-pentylamine and all other isomers), trihexylamines (including tri-n-hexylamine and all other isomers), triheptylamines (including tri-n-heptylamine and all other isomers), trioctylamines (including tri-n-octylamine and all other isomers), trinonylamines (including tri-n-nonylamine and all other isomers), tridecylamines (including tri-n-decylamine and all other isomers), tridodecylamine (including tri-n-dodecylamine and all other isomers), tritetradecylamines (including tri-n-tetradecylamine and all other isomers), tripentadecylamine (including tri-n-pentadecylamine and all other isomers), trihexadecyclamine (including tri-n-hexadecylamine and all other isomers), tri(2-ethyl-n-hexyl)amine, N-dimethyl-decylamine (including N-dimethyl-n-decylamine and all other isomers), N-dimethyl-dodecylamine (including N-dimethyl-n-dodecylamine and all other isomers), N-dimethyl-tetradecylamine (including N-dimethyl-n-tetradecylamine and all other isomers), N-dioctyl-methylamine (including N-di-n-octyl-methylamine and all other isomers), N-dihexyl-methylamine (including N-dihexyl-methylamine and all other isomers), tricyclopentylamine, tricyclohexylamine, tricyclooctylamine and derivates thereof which are substituted by one or more methyl, ethyl, 1-propyl, 2-propyl, 1-butyl, 2-butyl or 2-methyl-2-propyl groups,
N-methyl-dicyclohexylamine, N-ethyldicyclohexylamine,
Triphenylamine, N-methyldiphenylamine, N-ethyldiphenylamine, N-propyldiphenylamine, N-butyladiphenylamine, N-2-ethylhexyldiphenylamine, N-dipropylphenylamine, N-dibutylphenylamine, N-bis(2-ethylhexyl)phenylamine, tribenzylamine, N-methyldibenzylamine, N-ethyldibenzylamine and derivates thereof which are substituted by one or more methyl, ethyl, 1-propyl, 2-propyl, 1-butyl, 2-butyl or 2-methyl-2-propyl groups.
N-Octylpyrollidine, N-Nonylpyrollidine, N-Decylpyrollidine, N-Dodecyclpyrollidine, N-Tetradecylpyrollidine, N-Pentadecylpyrollidine, N-Heptylpiperidine, N-Octylpiperidine, N-Nonylpiperidine, N-Decylpiperidine, N-Dodecyclpiperidine, N-Tetradecylpiperidine, N-Pentadecylpiperidine.

In case of the possibility of isomers of the tertiary amines (I) mentioned above, all of the isomers shall be included by the name of the generic terms.

It is naturally also possible to use mixtures of various tertiary amines (I) in the process of the invention.

In the process of the invention, particular preference is given to using a saturated amine of the general formula (I) and more particularly preferred a saturated amine (I) in which the radicals R¹ to R³ are selected independently from the group consisting of C₁-C₁₈-alkyl and C₅-C₈-cycloalkyl but in total R¹ to R³ together having at least 12 carbon atoms and not more than 32 carbon atoms.

Very particular preference is given to using an amine of the general formula (I) in which the radicals R¹ to R³ are selected independently from the group consisting of C₅-C₈-alkyl. In particular the tertiary amine (I) is a tripentylamine, a trihexylamine, a triheptylamine, a trioctylamine, N-methyldicyclohexylamine, a N-dioctylmethylamine and/or a N-dimethyldecylamine, whereby tri-n-pentylamine, tri-n-hexylamine, tri-n-heptylamine, tri-n-octylamine and N-dimethyl-n-decylamine are particularly preferred. Very particular preferred as tertiary amine (I) are tripentylamine, trihexylamine and/or a triheptylamine.

The amount of the tertiary amine (I) to be used in the hydrogenation process of the invention is generally from 0.05 to 0.99 mL tertiary amine (I) per mL of the total reactor volume and preferably from 0.2 to 0.95 mL tertiary amine (I) per mL of the total reactor volume, whereby the volume of the tertiary amine (I) is based on the volume of the liquid tertiary amine (I) it would have as pure substance under reaction conditions.

The term “reactor volume” according to the invention defines the volume of the empty reactor. The term “total reactor volume” defines the volume that is left in the reactor after the heterogeneous catalyst has been built in the reactor. Therefore, the term “total reactor volume” is equal to “reactor volume” minus “catalyst's volume”.

For the process of the invention it is crucial that the hydrogenation of carbon dioxide is carried out in the presence of a diamine (II). The addition of a diamine (II) leads to an increase of the space-time-yield and thereby to a more economic process.

The diamine (II) to be used in the hydrogenation step of the invention is preferably an amine of the general formula (IIa),

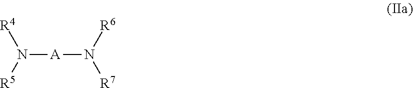

where
A is unsubstituted or at least monosubstituted methylene, ethylene, trimethylene, tetramethylene, pentamethylene or hexamethylene,
   where the substituents are selected from the group consisting of: F, Cl, Br, OR^{B}, OCOR⁸, COOR⁸ and C₁-C₁₀-alkyl,
   where R⁸ is selected from the group consisting of H and C₁-C₁₀-alkyl;
R⁴, R⁵, R⁶, R⁷ are identical or different and are each, independently of one another, an unbranched or branched, acyclic or cyclic, aliphatic, araliphatic or aromatic radical having from 1 to 46 carbon atoms, preferably from 1 to 18 carbon atoms, where individual carbon atoms can also be substituted, independently of one another, by a hetero group selected from the groups consisting of —O— and >N—,
   or two radicals R⁴, R⁵ can be joined to one another to form a chain comprising at least four atoms,
   and/or two radicals R⁶, R⁷ can be joined to one another to form a chain comprising at least four atoms,
   or two radicals R⁴, R⁶ can be joined to one another to form a chain comprising at least two atoms,
   and/or two radicals R⁵ and R⁷ can be joined to one another to form a chain comprising at least two atoms;
      or an amine of the general formula (IIb)

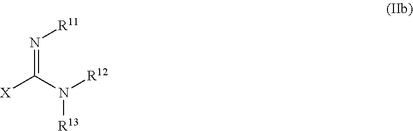

where
R¹¹ is H or unsubstituted or at least monosubstituted C₁-C₁₀-alkyl,
   where the substituents are selected from the group consisting of:
   F, Cl, Br, OR⁸, OCOR⁸, COOR⁸ and C₁-C₁₀-alkyl,
   where R⁸ is selected from the group consisting of H and C₁-C₁₀-alkyl, or
   R¹¹ is CR^{11a} and CR^{11a} is joined to CR^{12a} via a C—C-double-bond or via a methylene group,
   where R^{11a} is H or unsubstituted C₁-C₁₀-alkyl;
R¹² is H or unsubstituted or at least monosubstituted C₁-C₁₀-alkyl,
   where the substituents are selected from the group consisting of:
   F, Cl, Br, OR⁸, OCOR⁸, COOR⁸ and C₁-C₁₀-alkyl,
   where R⁸ is selected from the group consisting of H and C₁-C₁₀-alkyl, or
   R¹² is CR^{12a} and CR^{12a} is joined to CR^{11a} via a C—C-double-bond or vie a methylene group,
   where R^{12a} is H or unsubstituted C₁-C₁₀-alkyl;
R¹³ is H or unsubstituted or at least monosubstituted C₁-C₁₀-alkyl,
   where the substituents are selected from the group consisting of:
   F, Cl, Br, OR⁸, OCOR⁸, COOR⁸ and C₁-C₁₀-alkyl,
   where R⁸ is selected from the group consisting of H and C₁-C₁₀-alkyl, or
   R¹³ and R¹⁶ are joined to one another to form a bond or a chain comprising at least one atom;
X is H, NR¹⁴R¹⁵ or CR¹⁶R¹⁷,
   where
   R¹⁴, R¹⁵ are independently or one another H or unsubstituted or at least monosubstituted C₁-C₁₀-alkyl,
      where the substituents are selected from the group consisting of:
      F, Cl, Br, OR⁸, OCOR⁸, COOR⁸ and C₁-C₁₀-alkyl,
      where R⁸ is selected from the group consisting of H and C₁-C₁₀-alkyl;
   R¹⁶, R¹⁷ are independently or one another H or unsubstituted or at least monosubstituted C₁-C₁₀-alkyl,
      where the substituents are selected from the group consisting of:
      F, Cl, Br, OR⁸, OCOR⁸, COOR⁸ and C₁-C₁₀-alkyl,
      where R⁸ is selected from the group consisting of H and C₁-C₁₀-alkyl or
      R¹⁶ and R¹³ are joined to one another to form a bond or a chain comprising at least one atom.

The diamine (II) to be used in the hydrogenation step of the invention is more preferably an amine of the general formula (IIa),

where
A is unsubstituted methylene, ethylene, trimethylene, tetramethylene, pentamethylene or hexamethylene,
R⁴, R⁵, R⁶, R⁷ are identical or different and are each, independently of one another, an unbranched or branched, acyclic or cyclic, aliphatic, araliphatic or aromatic radical having from 1 to 46 carbon atoms, preferably from 1 to 18 carbon atoms, where individual carbon atoms can also be substituted, independently of one another, by a hetero group selected from the groups consisting of —O— and >N—,
   or
   two radicals R⁴, R⁵ can be joined to one another to form a chain comprising at least four atoms in and two radicals R⁶, R⁷ can be joined to one another to form a chain comprising at least four atoms,
   or
   two radicals R⁴, R⁶ can be joined to one another to form a chain comprising at least two atoms and two radicals R⁵ and R⁷ can be joined to one another to form a chain comprising at least two atoms;
      or an amine of the general formula (IIb)
      where
R¹¹ is H or unsubstituted C₁-C₁₀-alkyl,
   or
   R¹¹ is CR^{11a} and CR^{11a} is joined to CR^{12a} via a C—C-double-bond or via a methylene group,
   where R^{11a} is H;
R¹² is H or unsubstituted C₁-C₁₀-alkyl,
   or
   R¹² is CR^{12a} and CR^{12a} is joined to CR^{11a} via a C—C-double-bond or via a methylene group,
   where R^{12a} is H;
R¹³ is H or unsubstituted C₁-C₁₀-alkyl,
   or
   R¹³ and R¹⁶ are joined to one another to form a bond or a chain comprising at least one atom;
X is H, NR¹⁴R¹⁵ or CR¹⁶R¹⁷,
   where
   R¹⁴, R¹⁵ are independently or one another H or unsubstituted C₁-C₁₀-alkyl,
   R¹⁶, R¹⁷ are independently or one another H or unsubstituted C₁-C₁₀-alkyl, or
      R¹⁶ and R¹³ are joined to one another to form a bond or a chain comprising at least one atom.

In a case R⁴ and R⁵ are joined to one another in a preferred embodiment they form together with the nitrogen atom a pyrrolidine or a piperidine ring. In a case R⁶ and R⁷ are joined to one another in a preferred embodiment they form together with the nitrogen atom a pyrrolidine or a piperidine ring. In a very preferred embodiment R⁴ and R⁵ form together with the nitrogen atom a pyrrolidine or a piperidine ring and R⁶ and R⁷ form together with the nitrogen atom a pyrrolidine or a piperidine ring.

In a case R⁴ and R⁶ are joined to one another in a preferred embodiment they form together with the with the “N-A-N” moiety a piperazine ring. In this case A is ethylene and R⁴ and R⁶ are joined to one another and form an ethylene moiety.

In case R¹¹ is CR^{11a} and CR^{11a} is joined to CR^{12a} via a C—C-double-bond an imidazole ring is formed. In this case X is preferably H or CR¹⁶R¹⁷.

In case R¹⁶ and R¹³ are joined to one another they preferably form a bond, a methylene or an ethylene moiety. In this case CR^{11a} is preferably joined to CR^{12a} via a methylene group to form a six-membered ring.

Very particular preference is given to using diamines (II) selected from the group consisting of N,N,N′,N′-tetramethyl-ethane-1,2-diamine (TMEDA), N,N,N′,N′-tetramethyl-butane-1,4-diamine, pentamethylenedipiperidine (1,1′-(1,5-pentanediyl)bis-piperidine), tetramethylenedipyrrolidine (1,1′-(1,4-butanediyl)bis-pyrrolidine), 1,8-diaza-bicylo[5.4.0]undec-7-ene (DBU), 1,5-diazabicyclo[4.3.0]non-5-ene (DBN), bicyclo[2.2.2.]-1,4-diazooctane (DABCO), 1-methylimidazole, 1,2-dimethylimidazole, guanidine, guanidiencarbonate, tert-butyltetramethylguanidine (2-tert-Butyl-1,1,3,3-tetramethylguanidine) and tetramethylguanidine (1,1,3,3-tetramethylguanidine).

Methylene has the structure (—CH₂—), ethylene has the structure (—CH₂CH₂—), trimethylene has the structure (—CH₂CH₂CH₂—), tetramethylene has the structure (—CH₂CH₂CH₂CH₂—), pentamethylene has the structure (—CH₂CH₂CH₂CH₂CH₂—) and hexamethylene has the structure (—CH₂CH₂CH₂CH₂CH₂CH₂—).

Within the context of the present invention, C₁-C₁₀-alkyl are understood as meaning branched, unbranched, saturated and unsaturated groups. Preference is given to alkyl groups having 1 to 6 carbon atoms (C₁-C₆-alkyl). More preference is given to alkyl groups having 1 to 4 carbon atoms (C₁-C₄-alkyl).

Examples of saturated alkyl groups are methyl, ethyl, n-propyl, isopropyl, n-butyl, isobutyl, sec-butyl, tert-butyl, amyl and hexyl.

Examples of unsaturated alkyl groups (alkenyl, alkynyl) are vinyl, allyl, butenyl, ethynyl and propynyl.

The C₁-C₁₀-alkyl group can be unsubstituted or substituted with one or more substituents selected from the group F, Cl, Br, hydroxy (OH), C₁-C₁₀-alkoxy, C₅-C₁₀-aryloxy, C₅-C₁₀-alkylaryloxy, C₅-C₁₀-heteroaryloxy comprising at least one heteroatom selected from N, O, S, oxo, C₃-C₁₀-cycloalkyl, phenyl, C₅-C₁₀-heteroaryl comprising at least one heteroatom selected from N, O, S, C₅-C₁₀-heterocyclyl comprising at least one heteroatom selected from N, O, S, naphthyl, amino, C₁-C₁₀-alkylamino, arylamino, C₅-C₁₀-heteroarylamino comprising at least one heteroatom selected from N, O, S, C₁-C₁₀-dialkylamino, C₁₀-C₁₂-diarylamino, C₁₀-C₂₀-alkylarylamino, C₁-C₁₀-acyl, C₁-C₁₀-acyloxy, NO₂, C₁-C₁₀-carboxy, carbamoyl, carboxamide, cyano, sulfonyl, sulfonylamino, sulfinyl, sulfinylamino, thiol, C₁-C₁₀-alkylthiol, C₅-C₁₀-arylthiol or C₁-C₁₀-alkylsulfonyl.

In case of the possibility of isomers of the diamines (II) mentioned above, all of the isomers shall be included by the name of the generic terms.

It is naturally also possible to use mixtures of various diamines (II) in the process of the invention.

The amount of the diamine (II) to be used in the hydrogenation process of the invention is generally from 0.001 to 0.01 mL diamine (II) per mL of the total reactor volume and preferably from 0.001 to 0.2 mL diamine (II) per mL of the total reactor volume, whereby the volume of the diamine (II) is based on the volume of the liquid diamine (II) it would have as pure substance under reaction conditions.

The carbon dioxide to be used in the hydrogenation of carbon dioxide can be used in solid, liquid or gaseous form. It is also possible to use industrially available gas mixtures comprising carbon dioxide. The hydrogen to be used in the hydrogenation of carbon dioxide is generally gaseous. Carbon dioxide and hydrogen can also comprise inert gases such as nitrogen or noble gases, but surprisingly, the gold catalysts are also tolerating carbon monoxide, which is a catalyst poison when using the standard ruthenium catalysts for this reaction. However, the content of these gases, especially carbon monoxide, should not exceed 20 mol-% based on the total amount of carbon dioxide and hydrogen in the hydrogenation reactor. Although larger amounts may likewise be tolerable, they generally require the use of higher pressure in the reactor which in turn makes further compression energy necessary.

The hydrogenation of carbon dioxide is carried out in the liquid phase at a temperature of from 0 to 200° C. and a total pressure of from 0.2 to 30 MPa abs. The temperature is preferably at least 20 C°, more preferably at least 30° C. and also preferably not more than 100° C. The total pressure is preferably at least 1 MPa abs and particularly preferably at least 5 MPa and also generally not more than 25 MPa abs and preferably not more than 20 MPa abs.

The molar ratio of hydrogen to carbon dioxide in the feed to the hydrogenation reactor is preferably from 0.1 to 10 and particularly preferably from 1 to 3.

The molar ratio of carbon dioxide to tertiary amine (I) in the feed to the hydrogenation reactor is generally from 0.1 to 20 and preferably from 0.5 to 3.

The molar ratio of diamine (II) to tertiary amine (I) in the feed of the hydrogenation reactor is generally from 0.001 to 0.2 and preferably from 0.005 to 0.05.

The hydrogenation is carried out in the presence of a polar solvent. We have found that by the use of polar solvent higher space-time-yields are achieved. The molar ratio of polar solvent to tertiary amine (I) in the feed to the hydrogenation reactor is generally from 0.01 to 20 and preferably from 1 to 10.

In a preferred embodiment in the process of the invention, at least one polar solvent selected from the group consisting of methanol, ethanol, 1-propanol, 2-propanol, 1-butanol, 2-butanol, 2-methyl-1-propanol and water is used in the hydrogenation of carbon dioxide.

It is naturally also possible to use mixtures of various polar solvents in the process of the invention.

As hydrogenation reactors, it is in principle possible to use all reactors which are suitable in principle for heterogeneously catalyzed gas/liquid reactions at the given temperature and the given pressure. Suitable standard reactors for the hydrogenation are indicated, for example, in K. D. Henkel, “Reactor Types and Their industrial Applications”, in Ullmann's Encyclopedia of Industrial Chemistry, 2005, Wiley-VCH Verlag GmbH & Co. KGaA, DOI: 10.1002/14356007.b04_{—}087. Examples which may be mentioned are stirred tank reactors, tubular reactors, multi-tubular reactors, multi-channel reactors, micro-channel reactors or fixed bed reactors.

The hydrogenation of carbon dioxide in the process of the invention can be carried out batchwise or continuously. In the case of batch operation, the hydrogenation reactor is typically charged with the heterogeneous catalyst and the desired tertiary amine (I), the diamine (II), the polar solvent and carbon dioxide and hydrogen subsequently introduced to the desired pressure at the desired temperature. After the hydrogenation, the reactor is generally depressurized and the liquid reaction mixture separated from the heterogeneous catalyst.

In the continuous mode of operation, the tertiary amine (I), the diamine (II), the polar solvent, carbon dioxide and hydrogen are introduced continuously. However, if a fixed-bed heterogeneous catalyst is used, it is generally present beforehand in fixed form in the reactor. In case of a suspended heterogeneous catalyst, it normally might also be present in the reactor beforehand or be introduced in an amount equal to that of its removal by the continuous reactor discharge. Accordingly, the liquid reaction mixture is continuously discharged from the reactor so that the average liquid level in the reactor remains constant. Preference is given to the continuous hydrogenation of carbon dioxide.

Irrespective of the type of the heterogeneous catalyst and whether the hydrogenation is performed batchwise or continuously, the liquid reaction mixture is after the hydrogenation reaction generally separated from the heterogeneous catalyst. In case of using a fixed-bed catalyst, it normally stays in the reactor when the reaction mixture is discharged, due to its immobilization. In case of using a non-immobilized heterogeneous catalyst, it is typically either kept back in the reactor by common precautions (e.g. by a mesh or a filter at the outlet) or separated from the reaction mixture by simple filtration, decantation or centrifugation and recycled back to the hydrogenation reactor. After the separation of the catalyst, the liquid reaction mixture is practically free of gold, which means 1 wt.-ppm of gold or less in the separated liquid reaction mixture.

The average residence time in the reactor is generally from 10 minutes to 10 hours.

The obtained liquid reaction mixture generally comprises formic acid, the tertiary amine (I), the diamine (II) and the polar solvent. The liquid reaction mixture generally contains formic acid and the tertiary amine (I) in form of a formic acid/amine adduct. If a tertiary amine of formula (I) was used, the formic acid/amine adduct usually has the general formula (III)

xHCOOH*NR¹R²R³ (III)

where the radicals R¹ to R³ are the radicals described for the tertiary amine (I) and x is from 0.5 to 5, preferably from 1.2 to 2.6. The factor x can be determined, for example by titration with KOH solution against phenolphthalein. The precise composition of the formic acid/amine adduct (III) depends on many parameters, for example the prevailing concentrations of formic acid and tertiary amine (I), pressure, temperature or the presence and nature of further components, in particular of polar solvents present. The composition of the formic acid/amine adduct (III) can therefore also change over the individual process steps in which the formic acid/amine adduct (III) is in each case referred to in the present patent application. The composition of the formic acid/amine adduct (III) can easily be determined in each process step by determining the formic acid content by acid-base titration and determining the amine content by gas chromatography.

The liquid reaction mixture generally contains the diamine (II) in form of a formic acid salt.

A further object of the present invention is a process, wherein the liquid reaction mixture obtained by the hydrogenation comprises formic acid and tertiary amine (I) in form of a formic acid/amine adduct (III), diamine (II) and the polar solvent.

From the liquid reaction mixture obtained in the hydrogenation reactor, in a preferred embodiment, the polar solvent is separated off in a first distillation apparatus.

A distillate (D1) and a bottoms mixture (S1) are obtained from the first distillation apparatus. The distillate (D1) comprises the polar solvent which has been separated off and is, in a preferred embodiment, recirculated to the hydrogenation reactor. The bottoms mixture (S1) comprises the tertiary amine (A1), the formic acid/amine adduct (III) and the diamine (II). In an embodiment of the process of the invention, the polar solvent is partly separated off in the first distillation apparatus so that the bottoms mixture (S1) still comprises polar solvent which has not yet been separated off. It is possible to separate off, for example, from 5 to 98% by weight of the polar solvent comprised in the liquid reaction mixture, with preference being given to from 50 to 98% by weight, more preferably from 80 to 98% by weight and particularly preferably from 80 to 90% by weight, being separated off, in each case based on the total weight of the polar solvent comprised in the liquid reaction mixture.

In a further embodiment of the process of the invention, the polar solvent is completely separated off in the first distillation apparatus. For the purposes of the present invention, “completely separated off” means a removal of more than 98% by weight of the polar solvent comprised in the liquid reaction mixture, preferably more than 98.5% by weight, particularly preferably more than 99% by weight, in particular more than 99.5% by weight, in each case based on the total weight of the polar solvent comprised in the liquid reaction mixture.

The distillate (D1) which has been separated off in the first distillation apparatus is, in a preferred embodiment, recirculated to the hydrogenation reactor.

A further object of the present invention is a process, wherein the polar solvent is separated off as a distillate (D1) in a first distillation apparatus and the obtained bottoms mixture (S1) comprises the formic acid/amine adduct (III) and possibly the free tertiary amine (I).

The separation of the polar solvent from the liquid reaction mixture can, for example, be carried out in an evaporator or in a distillation unit comprising a vaporizer and column, with the column being provided with ordered packing, random packing elements and/or trays.

The at least partial removal of the polar solvent is preferably carried out at a temperature at the bottom at which no free formic acid is formed from the formic acid/amine adduct (III) at the given pressure. The factor xᵢ of the formic acid/amine adduct (III) in the first distillation apparatus is generally in the range from 0.4 to 3, preferably in the range from 0.6 to 1.8, particularly preferably in the range from 0.7 to 1.7.

In general, the temperature at the bottom of the first distillation apparatus is at least 20° C., preferably at least 50° C. and particularly preferably at least 70° C., and generally not more than 210° C., preferably not more than 190° C. The temperature in the first distillation apparatus is generally in the range from 20° C. to 210° C., preferably in the range from 50° C. to 190° C. The pressure in the first distillation apparatus is generally at least 0.001 MPa abs, preferably at least 0.005 MPa abs and particularly preferably at least 0.01 MPa abs, and generally not more than 1 MPa abs and preferably not more than 0.1 MPa abs. The pressure in the first distillation apparatus is generally in the range from 0.0001 MPa abs to 1 MPa abs, preferably in the range from 0.005 MPa abs to 0.1 MPa abs and particularly preferably in the range from 0.01 MPa abs to 0.1 MPa abs.

In the removal of the polar solvent in the first distillation apparatus, the formic acid/amine adduct (III) and free tertiary amine (I) can be obtained at the bottom of the first distillation apparatus, since formic acid/amine adducts having a low amine content are formed during the removal of the polar solvent. As a result, a bottoms mixture (S1) comprising the formic acid/amine adduct (III) and the free tertiary amine (I) is formed. The bottoms mixture (S1) comprises, depending on the amount of polar solvent separated off, the formic acid/amine adduct (III) and possibly the free tertiary amine (I) formed in the liquid phase of the first distillation apparatus. The bottoms mixture (S1) is optionally worked up further.

A further object of the present invention is a process, wherein the bottoms mixture is fed to a second distillation apparatus wherein the formic acid is released from the formic acid/amine adduct (III), and a bottom product is obtained comprising tertiary amine (I) and diamine (II).

It is also possible to feed the liquid reaction mixture from the hydrogenation reactor, directly to the second distillation apparatus, without separating off the polar solvent.

Preferably the polar solvent is separated off and the obtained bottoms mixture (S1) is then subjected to distillation in a second distillation apparatus, in which formic acid is released from the formic acid/amine adduct (III) by thermal dissociation and removed. This step can generally be carried out under process parameter known in the prior art for the thermal dissociation of formic acid/amine adducts into free formic acid and the respective amine and, for example, described in EP 0 181 078 A or WO 2006/021,411.

The second distillation apparatus generally comprises, in addition to the actual column body with internals, inter alia a top condenser and a bottom evaporator. In addition, this may optionally also comprise still further peripheral apparatuses or internals and, for example, a flash container in the feed (for example for separating gas and liquid in the feed to the column body), an intermediate evaporator (for example for improved heat integration of the process) or internals for avoiding or reducing aerosol formation (such as, for example, thermostatable trays, demisters, coalescers or deep-bed diffusion filters). The column body may be equipped, for example, with structured packings, random packings or trays. The number of separation stages required is dependent in particular on the type of tertiary amine (I), the concentration of formic acid and tertiary amine (I) in the bottoms mixture (S1) fed to the second distillation apparatus and the desired concentration or the desired purity of the formic acid and can be determined by the person skilled in the art in the customary manner. In general, the number of required separation stages is ≧3, preferably ≧6 and particularly preferably ≧7. There are in principle no upper limits. For practical reasons, however, it is likely to be customary to use as a rule ≦50, optionally ≦30, separation stages.

The bottoms mixture (S1) can be fed to the second distillation apparatus, for example, as a side stream to the column body.

Optionally, the addition can also be effected upstream of a flash evaporator, for example. In order to keep the thermal load on the feed stream in the distillation apparatus as low as possible, it is generally advantageous rather to feed this to the lower region of the distillation apparatus. Thus, it is preferable to feed in the product mixture in the region of the lower fourth, preferably in the region of the lower fifth and particularly preferably in the region of the lower sixth of the available separation stages, a direct feed into the bottom of course also being included here.

Alternatively, however, it is also preferable to feed said bottoms mixture (S1) to the bottom evaporator of the second distillation apparatus.

The second distillation apparatus is generally operated at a bottom temperature of from 100 to 300° C. and a pressure of from 30 to 3000 hPa abs. Preferably, the second distillation apparatus is operated at a bottom temperature of ≧120° C., particularly preferably of ≧140° C. and preferably of ≦220° C. and particularly preferably of ≦200° C. The pressure is preferably ≧30 hPa abs, particularly preferably ≧60 hPa abs and preferably ≦1500 hPa abs and particularly preferably ≦500 hPa abs.

The formic acid released by the thermal dissociation can be obtained as top product and/or side product from the second distillation apparatus. When the bottoms mixture (S1) comprises constituents boiling lower than formic acid, it may be advantageous to separate these off by distillation as top product and the formic acid in the side take-off. Where gases may be dissolved in the bottoms mixture (S1) (such as, for example, carbon monoxide or carbon dioxide), however, it is as a rule also possible to separate off the formic acid together with these as top product. If the bottoms mixture (S1) comprises constituents boiling higher than formic acid, formic acid is preferably separated off by distillation as top product, but optionally instead of these or in addition in the form of a second stream in the side take-off. The constituents boiling higher than formic acid are in this case then preferably taken off via an additional side stream.

In this way, formic acid having a content of up to 100 wt.-% can be obtained. In general, formic acid contents of from 75 to 99.995 wt.-% are achievable without problems. The residual content to 100 wt.-% might, for example, be water added to the hydrogenation of carbon dioxide to promote the heterogeneously catalyzed reaction. Thus, water may already be present in the bottoms mixture (S1) fed to the second distillation apparatus but may optionally also form only during the thermal separation in small amounts as a result of decomposition of formic acid itself.

In the recovery of concentrated formic acid having a content from 95 to 100 wt.-% as bottom or side product, water is discharged with a part of the eliminated formic acid in a side stream. The formic acid content of this side stream is typically from 75 to 95 wt.-%. However, it is also possible to discharge the water and the eliminated formic acid in a common top or side stream. The formic acid content of the product thus obtained is then as a rule from 85 to 95 wt.-%.

The formic acid obtainable by the process according to the invention has a low color number and a high color number stability. In general, a color number of ≦20 APHA and in particular even of ≦10 APHA and optionally even of ≦5 APHA can be achieved without problems. Even on storage for several weeks, the color number remains virtually constant or increases only insignificantly.

The bottom product obtained in the step of the removal of formic acid by distillation containing tertiary amine (I) and the diamine (II) is advantageously recycled to the hydrogenation reactor. In general, from 10 to 100%, preferably from 50 to 100%, particularly preferably from 80 to 100%, very particularly preferably from 90 to 100% and in particular from 95 to 100% of the tertiary amine (II) of the bottom product is recycled to the step of the hydrogenation.

The bottom product taken off from the second distillation apparatus can still comprise small residual amounts of formic acid, but the molar ratio of formic acid to tertiary amine (I) is preferably ≦0.1 and particularly preferably ≦0.05.

DE 34 28 319 A has described the thermal dissociation of an adduct of formic acid and a tertiary amine having C₆-C₁₄-alkyl radicals in a dissociation column. Likewise, WO 2006/021,411 also describes the thermal dissociation of an adduct of formic acid and a tertiary amine having a boiling point at atmospheric pressure of from 105 to 175° C. in a dissociation column. EP 0 563 831 A similarly discloses the thermal dissociation of an adduct of formic acid and a tertiary amine having a boiling point higher than that of formic acid, with added formamide being said to give a particularly color-stable formic acid.

The invention is illustrated by the following drawings and examples without being limited thereto.

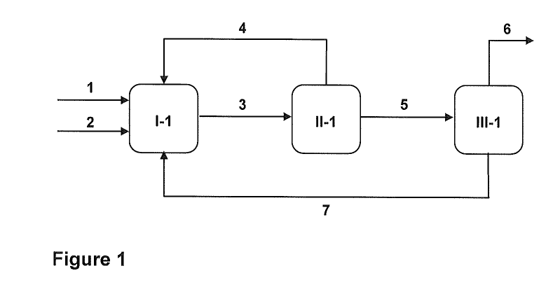

FIG. 1 shows a schematic block diagram of a possible embodiment of the process of the invention. In FIG. 1 the reference numerals have the following meanings:
I-1 hydrogenation reactor
II-1 first distillation apparatus
III-1 second distillation apparatus
1 stream comprising carbon dioxide
2 stream comprising hydrogen
3 stream comprising liquid reaction mixture
4 stream comprising polar solvent; (distillation (D1))
5 stream comprising bottoms mixture (S1)
6 stream comprising formic acid
7 stream comprising bottom product

In the embodiment of FIG. 1, stream 1 comprising carbon dioxide and stream 2 comprising hydrogen are fed to a hydrogenation reactor I-1. It is possible to feed further streams (not shown) to the hydrogenation reactor I-1 in order to compensate any losses of tertiary amine (I), diamine (II) or heterogeneous catalyst.

In the hydrogenation reactor I-1, carbon dioxide and hydrogen are reacted in the presence of a tertiary amine (I), diamine (II), polar solvent and a heterogeneous catalyst comprising gold. This gives a liquid reaction mixture which comprises the tertiary amine (I), the diamine (II), the polar solvent and the formic acid/amine adduct (III). The liquid reaction mixture is fed as stream 3 to the first distillation apparatus II-1. In the first distillation apparatus II-1 the liquid reaction mixture is separated into a distillate (D1) comprising the polar solvent, which is recirculated as stream 4 to the hydrogenation reactor I-1 and a bottoms mixture (S1).

The bottoms mixture (S1) comprises the tertiary amine (I), the diamine (II) and the formic acid/amine adduct (III). The bottoms mixture (S1) is fed as stream 5 to the second distillation apparatus III-1.

The formic acid/amine adduct (III) comprised in the bottoms mixture (S1) is dissociated into formic acid and free tertiary amine (I) in the second distillation apparatus III-1. At the top of the second distillation apparatus III-1 formic acid is discharged as stream 6 from the second distillation apparatus III-1. The bottom product comprising the tertiary amine (I) and the diamine (II) is recirculated as stream 7 to the hydrogenation reactor I-1.

### EXAMPLES

### Materials

Unless stated otherwise, the following specific materials were used.
a) Au on ZrO₂ was synthesized with different gold contents according to a literature procedure: Q. Y. Bi, X. L. Du, Y. M. Liu, Y. Cao, H. Y. He, K. N. Fan, J. Am. Chem. Soc. 2012, 134, 8926-8933.
b) AUROlite™ Au/TiO₂:
   1.0±0.1 wt.-% Au on TiO₂ extrudates of 1.5 mm diameter and an average length of 5 mm, bulk density 0.85-0.95 g/mL, BET surface 40-50 m2/g. Supplied by Strem Chemicals Inc. Used as received.

Examples without addition of a diamine (II) are shown in table 1:

TABLE 1

| | | | T | p | t | % | Act* |
|---|---|---|---|---|---|---|---|
| Catalyst | Solvent | Amine | [° C.] | [bar] | [h] | FA | g/gh |
| TiO₂^{a)} | MeOH/H₂O | NHex₃ | 40 | 180 | 10 | 2.1 | 0.01 |
| (1% Au) | | | | | | | |
| TiO₂^{a)} | MeOH/H₂O | NHex₃ | 70 | 180 | 10 | 1.2 | 0.01 |
| (1% Au) | | | | | | | |
| TiO₂^{a)} | — | NHex₃ | 40 | 180 | 10 | 0.8 | 0.01 |
| (1% Au) | | | | | | | |
| TiO₂^{a)} | MeOH | NHex₃ | 40 | 200 | 10 | 2.2 | 0.01 |
| (1% Au) | | | | | | | |
| TiO₂^{a)} | MeOH | NHex₃ | 55 | 200 | 2.5 | 0.6 | 0.04 |
| (1% Au) | | | | | | | |
| TiO₂^{a)} | MeOH | NHex₃ | 70 | 200 | 2 | 0.5 | 0.01 |
| (1% Au) | | | | | | | |

FA = Formic Acid; [FA]/g[Catalyst.]h; Aurolite Catalyst (1% Au on TiO₂); MeOH: methanol; NHex₃: tri-n-hexylamine; Reaction carried out in a 250 mL HC4-reactor, 5 g catalyst were used, the reaction mixture was pressurized with 30 g CO₂ and the pressure was then raised with H₂ to the pressure given in the table; 100 g reaction mixture were used; when MeOH was added, 20 g MeOH and 80 g NHex₃ were used.
^{a)} = extrudates
Act* g/gh is defined as the amount [in g] of formic acid which is produced by the amount of catalyst [in g] per hour.

Examples with diamines (II) as additives are shown in table 2:

| Catalyst | Solvent | Amine | Diamine | [%] | T [° C.] | p [bar] | t [h] | % FA | Act* g/gh |
|---|---|---|---|---|---|---|---|---|---|
| TiO₂^{a)} | MeOH | NHex₃ | methylimidazole (1) | 1 | 40 | 200 | 10 | 4.4 | 0.02 |
| (1% Au) | | | | | | | | | |
| TiO₂^{a)} | MeOH | NHex₃ | 1,2-dimethyl | 1 | 50 | 200 | 10 | 2.1 | 0.01 |
| (1% Au) | | | imidazole | | | | | | |
| TiO₂^{a)} | MeOH | NHex₃ | TMEDA | 1 | 50 | 200 | 10 | 6.8 | 0.06 |
| (1% Au) | | | | | | | | | |
| TiO₂^{a)} | MeOH | NHex₃ | pentamethylene | 1 | 50 | 200 | 10 | 6.7 | 0.08 |
| (1% Au) | | | dipiperidine | | | | | | |
| TiO₂^{a)} | MeOH | NHex₃ | DBU | 1 | 50 | 200 | 10 | 8.2 | 0.13 |
| (1% Au) | | | | | | | | | |
| TiO₂^{a)} | MeOH | NHex₃ | 2,2-dimethyl-1,3- | 1 | 50 | 200 | 10 | 3.0 | 0.01 |
| (1% Au) | | | propanediamine | | | | | | |
| TiO₂^{a)} | MeOH | NHex₃ | tetramethylene | 1 | 50 | 200 | 10 | 8.7 | 0.16 |
| (1% Au) | | | dipyrollidine | | | | | | |
| TiO₂^{a)} | MeOH | NHex₃ | pentamethylene | 1 | 70 | 200 | 1 | 4.1 | 0.18 |
| (1% Au) | | | dipiperidine | | | | | | |
| TiO₂^{a)} | MeOH | NHex₃ | pentamethylene | 1 | 40 | 200 | 1 | 2.1 | 0.05 |
| (1% Au) | | | dipiperidine | | | | | | |
| TiO₂^{a)} | MeOH | NHex₃ | TMEDA | 1 | 70 | 200 | 1 | 2.8 | 0.08 |
| (1% Au) | | | | | | | | | |
| TiO₂^{a)} | MeOH | NHex₃ | DBU | 0.4 | 70 | 130 | 3 | 4.6 | 0.09 |
| (1% Au) | | | | | | | | | |
| ZrO₂^{b)} | MeOH | NHex₃ | tetramethylene | 1 | 70 | 200 | 3 | 8.3 | 0.79 |
| (3.8% Au) | | | dipyrollidine | | | | | | |
| ZrO₂^{b)} | MeOH | NHex₃ | DBU | 1 | 50 | 200 | 10 | 6.8 | 0.1 |
| (2.3% Au) | | | | | | | | | |
| ZrO^{b)} | MeOH | NHex₃ | tetramethylene | 1 | 50 | 200 | 10 | 13.4 | 0.45 |
| (4% Au) | | | dipyrollidine | | | | | | |
| ZrO₂^{b)} | MeOH | NHex₃ | tetramethylene | 1 | 50 | 200 | 10 | 6.7 | 0.08 |
| (2.3% Au) | | | dipyrollidine | | | | | | |
| TiO₂^{a)} | MeOH | NHex₃ | guanidincarbonate | 1 | 70 | 210 | 2 | 3.1 | 0.06 |
| (1% Au) | | | | | | | | | |
| TiO₂^{a)} | MeOH | NHex₃ | tert | 1 | 70 | 211 | 2 | 2.9 | 0.05 |
| (1% Au) | | | butyltetramethyl | | | | | | |
| | | | guanidine | | | | | | |
| TiO₂^{a)} | MeOH | NHex₃ | tetramethyl | 1 | 70 | 205 | 2 | 5.4 | 0.10 |
| (1% Au) | | | guanidine | | | | | | |
| TiO₂^{a)} | MeOH | NHex₃ | tetramethylene | 1 | 70 | 194 | 2 | 7.2 | 0.34 |
| (1% Au) | | | butandiamine | | | | | | |

DBU: Diazabicycloundecane;
TMEDA: Tetramethylenediamine;
MeOH: methanol;
NHex₃: tri-n-hexylamine; ZrO₂: Gold on ZrO₂ was synthesized according to a literature procedure: Q.Y. Bi, X.L. Du, Y.M. Liu, Y. Cao, H.Y. He, K.N. Fan, J. Am. Chem. Soc. 2012, 134, 8926-8933, FA = Formic Acid; [FA]/g[Catalyst.]h; Aurolite Catalyst (1% Au on TiO₂); Reaction carried out in a 300 mL HC4-reactor, 5 g catalyst were used, the reaction mixture was pressurized with 30 g CO₂ and the pressure was then raised with H₂ to the pressure given in the tabl; 20 g MeOH and 79 g NHex₃ were used. Act* g/gh is defined as the amount [in g] of formic acid which is produced by the amount of catalyst [in g] per hour.
^{a)}= extrudates;
^{b)}= powder

1-15. (canceled)

16. A process for preparing formic acid by hydrogenation of carbon dioxide in the presence of a tertiary amine (I), a diamine (II), a polar solvent and a catalyst comprising gold at a pressure of from 0.2 to 30 MPa abs and a temperature of from 0 to 200° C., wherein the catalyst is a heterogeneous catalyst comprising gold.

17. The process of claim 16, wherein the heterogeneous catalyst comprising gold is a supported catalyst.

18. The process of claim 17, wherein the supported heterogeneous catalyst comprises silicon dioxide, aluminium oxide, zirconium oxide, magnesium oxide and/or titanium oxide as support.

19. The process of claim 17, wherein the supported heterogeneous catalyst comprises 0.1 to 20 wt.-% gold based on the total mass of the supported catalyst.

20. The process of claim 16, wherein the tertiary amine (I) comprises at least 12 carbon atoms.

21. The process of claim 16, wherein the tertiary amine (I) is tripentylamine, trihexylamine and/or a triheptylamine.

22. The process of claim 16, wherein the diamine is an amine of the general formula (IIa), 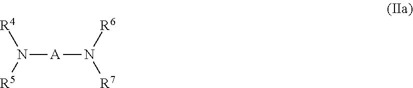
where
A is methylene, ethylene, trimethylene, tetramethylene, pentamethylene or hexamethylene, each of which is unsubstituted or at least monosubstituted with F, Cl, Br, OR⁸, OCOR⁸, COOR⁸ or C₁-C₁₀-alkyl, where R⁸ is selected from the group consisting of H and C₁-C₁₀-alkyl;
R⁴, R⁵, R⁶, R⁷ are, independently on each occurrence, an unbranched or branched, acyclic or cyclic, aliphatic, araliphatic or aromatic radical having from 1 to 46 carbon atoms, where individual carbon atoms are optionally substituted, independently of one another, by a hetero group selected from the group consisting of —O— and >N—,
or two radicals R⁴, R⁵ are optionally joined to one another to form a chain comprising at least four atoms,
and/or two radicals R⁶, R⁷ are optionally joined to one another to form a chain comprising at least four atoms,
or two radicals R⁴, R⁶ are optionally joined to one another to form a chain comprising at least two atoms,
and/or two radicals R⁵ and R⁷ are optionally joined to one another to form a chain comprising at least two atoms;
or an amine of the general formula (IIb) 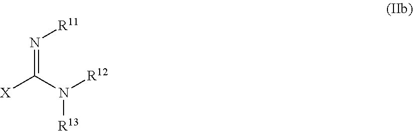
where
R¹¹ is H or C₁-C₁₀-alkyl, unsubstituted or at least monosubstituted with F, Cl, Br, OR⁸, OCOR⁸, COOR⁸ or C₁-C₁₀-alkyl, where R⁸ is selected from the group consisting of H and C₁-C₁₀-alkyl,
or
R¹¹ is CR^{11a} and CR^{11a} is joined to CR^{12a} via a C—C-double-bond or via a methylene group, where R^{11a} is H or unsubstituted C₁-C₁₀-alkyl;
R¹² is H or C₁-C₁₀-alkyl, unsubstituted or at least monosubstituted with F, Cl, Br, OR⁸, OCOR⁸, COOR⁸ or C₁-C₁₀-alkyl, where R⁸ is selected from the group consisting of H and C₁-C₁₀-alkyl,
or
R¹² is CR^{12a} and CR^{12a} is joined to CR^{11a} via a C—C-double-bond or vie a methylene group, where R^{12a} is H or unsubstituted C₁-C₁₀-alkyl;
R¹³ is H or C₁-C₁₀-alkyl, unsubstituted or at least monosubstituted with F, Cl, Br, OR⁸, OCOR⁸, COOR⁸ or C₁-C₁₀-alkyl, where R⁸ is selected from the group consisting of H and C₁-C₁₀-alkyl,
or
R¹³ and R¹⁶ are joined to one another to form a bond or a chain comprising at least one atom;
X is H, NR¹⁴R¹⁵ or CR¹⁶R¹⁷,
where
R¹⁴, R¹⁵ are, independently on each occurrence, H or C₁-C₁₀-alkyl, unsubstituted or at least monosubstituted with F, Cl, Br, OR⁸, OCOR⁸, COOR⁸ or C₁-C₁₀-alkyl, where R⁸ is selected from the group consisting of H and C₁-C₁₀-alkyl;
R¹⁶, R¹⁷ are, independently on each occurrence, H or C₁-C₁₀-alkyl, unsubstituted or at least monosubstituted with F, Cl, Br, OR⁸, OCOR⁸, COOR⁸ and C₁-C₁₀-alkyl, where R⁸ is selected from the group consisting of H and C₁-C₁₀-alkyl or
R¹⁶ and R¹³ are joined to one another to form a bond or a chain comprising at least one atom.

23. The process of claim 16, wherein the diamine (II) is selected from the group consisting of N,N,N′,N′-tetramethyl-ethane-1,2-diamine (TMEDA), N,N,N′,N′-tetramethyl-butane-1,4-diamine, pentamethylenedipiperidine (1,1′-(1,5-pentanediyl)bis-piperidine), tetramethylenedipyrrolidine (1,1′-(1,4-butanediyl)bis-pyrrolidine), 1,8-diaza-bicylo[5.4.0]undec-7-ene (DBU), 1,5-diazabicyclo[4.3.0]non-5-ene (DBN), bicyclo[2.2.2]-1,4-diazooctane (DABCO), 1-methylimidazole, 1,2-dimethylimidazole, guanidine, guanidiencarbonate, tert-butyltetramethylguanidine (2-tert-Butyl-1,1,3,3-tetramethylguanidine) and tetramethylguanidine (1,1,3,3-tetramethylguanidine).

24. The process of claim 16, wherein the polar solvent is selected from the group consisting of methanol, ethanol, 1-propanol, 2-propanol, 1-butanol, 2-butanol, 2-methyl-1-propanol and water.

25. The process of claim 16, wherein the hydrogenation is carried out in a hydrogenation reactor, the diamine (II) and tertiary amine (I) are fed into the reactor at a feed point, and the molar ratio of diamine (II) to tertiary amine (I) at the feed point is from 0.001 to 0.2.

26. The process of claim 16, wherein the hydrogenation produces a liquid reaction mixture comprising formic acid and tertiary amine (I) as a formic acid/amine adduct (III), diamine (II) and the polar solvent.

27. The process of claim 16, wherein the polar solvent is separated off as a distillate (D1) in a first distillation apparatus and an obtained bottoms mixture (S1) comprises the formic acid/amine adduct (III) and optionally the free tertiary amine (I).

28. The process of claim 27, wherein the bottoms mixture (S1) is fed to a second distillation apparatus wherein the formic acid is released from the formic acid/amine adduct (III), and a bottom product is obtained comprising tertiary amine (I) and diamine (II).

29. The process of claim 27, wherein the distillate (D1) is recirculated to the hydrogenation reactor.

30. The process of claim 28, wherein the bottom product is recirculated to the hydrogenation reactor.

31. The process of claim 22, wherein
R⁴, R⁵, R⁶, R⁷ are, independently on each occurrence, an unbranched or branched, acyclic or cyclic, aliphatic, araliphatic or aromatic radical having from 1 to 18 carbon atoms, where individual carbon atoms are optionally substituted, independently of one another, by a hetero group selected from the group consisting of —O— and >N—,
or two radicals R⁴, R⁵ are optionally joined to one another to form a chain comprising at least four atoms,
and/or two radicals R⁶, R⁷ are optionally joined to one another to form a chain comprising at least four atoms,
or two radicals R⁴, R⁶ are optionally joined to one another to form a chain comprising at least two atoms,
and/or two radicals R⁵ and R⁷ are optionally joined to one another to form a chain comprising at least two atoms.
